(19) United States
(12) Patent Application Publication
(10) Pub. No.: US 2025/0154216 A1
(43) Pub. Date: May 15, 2025

(54) IL-2 AND TL1A FUSION PROTEINS AND METHODS OF USE THEREOF

(71) Applicant: UNIVERSITY OF MIAMI, Miami, FL (US)

(72) Inventors: Robert B. LEVY, Miami, FL (US); Dietlinde WOLF, Miami, FL (US)

(21) Appl. No.: 18/838,917

(22) PCT Filed: Feb. 16, 2023
(86) PCT No.: PCT/US2023/062754

§ 371 Date: Aug. 15, 2024

Related U.S. Application Data
(60) Provisional application No. 63/310,726, filed on Feb. 16, 2022.

(51) Int. Cl. C07K 14/55; A61K 38/00; A61P 37/06; C07K 14/525

(52) U.S. Cl. CPC C07K 14/55; A61P 37/06; C07K 14/525; A61K 38/00; C07K 2319/30

(57) ABSTRACT

The present disclosure relates to chimeric polypeptides and methods of use thereof. The chimeric polypeptides comprise an interleukin-2 (IL-2) peptide; an immunoglobulin peptide; and a TL1 A peptide. The fusion proteins disclosed herein are a major advance and combine both TL1A (the physiologic ligand of TNFRSF25) and IL-2 (the physiologic ligand of CD25). Such a fusion protein can prolong the half-life of IL-2 and allow lower TLI1 levels. Thus, the inventors generated an !L-2-1gG1-TL1A fusion protein and tested its efficacy in vitro and in vivo. In vitro findings demonstrated that both the TL1A and IL-2 FP subunits were functional.

### CROSS-REFERENCE TO RELATED APPLICATIONS

This application claims the priority benefit of U.S. Provisional Application No. 63/310,726 filed Feb. 16, 2022, which is expressly incorporated herein by reference in its entirety.

### FIELD

The present disclosure relates to chimeric polypeptides and methods of use thereof.

### BACKGROUND

Tregs are a non-redundant CD4⁺ T cell population required for immune homeostasis/maintenance of peripheral tolerance. These cells suppress immune responses and inflammation and promote tissue repair, with clinical application to prevent liquid and solid organ graft rejection. Two major strategies to exploit Treg function are: 1) ex vivo expansion and subsequent cell therapy, or 2) in vivo expansion to increase numbers/function. Administration of low dose IL-2 can augment Tregs in situ and this approach is being tested in transplant and autoimmune studies. Importantly, ex vivo expansion is limited to Tregs from blood, whereas in vivo expansion can directly impact Tregs in target/diseased compartments, thus obviating trafficking requirements. What is needed are new polypeptides and compositions for expansion of Tregs in vivo.

The compounds, compositions, and methods disclosed herein address these and other needs.

### SUMMARY

Disclosed herein is a novel in vivo strategy targeting two Treg receptors: TNFRSF25 and CD25. It was shown that combining TL1A-IgG1 administration together with IL-2 has a dramatic effect, raising overall Treg levels up to 60% of CD4⁺ T cells in mouse peripheral blood. The inventors have shown the effect of TNFRSF25-+CD25 (IL2Ra) receptor stimulation on Tregs in hematopoietic stem cell transplants (HSCT) by manipulating donor Tregs and recipient Tregs in vivo.

The fusion proteins (FP) disclosed herein are a major advance and combine both TL1A (the physiologic ligand of TNFRSF25) and IL-2 (the physiologic ligand of CD25). Such a fusion protein can prolong the half-life of IL-2 and allow lower TL1A levels. Thus, the inventors generated an IL-2-IgG1-TL1A fusion protein and tested its efficacy in vitro and in vivo. In vitro findings demonstrated that both the TL1A and IL-2 FP subunits were functional. It was next found that in vivo administration of very low levels of the fusion proteins disclosed herein increased Treg frequency, even at two days post-injections.

In some aspects, disclosed herein is a chimeric polypeptide comprising:
an interleukin-2 (IL-2) peptide;
an IgG1 peptide; and
a TL1A peptide.

In some embodiments, the polypeptide further comprises a peptide linker between the IL-2 peptide and the IgG peptide. In some embodiments, the peptide linker is a 20 amino acid peptide linker. In some embodiments, the peptide linker is encoded by a nucleic acid at least 80% identical to SEQ ID NO:5 or a fragment thereof. In some embodiments, the peptide linker is encoded by SEQ ID NO:5.

In some embodiments, the interleukin-2 (IL-2) peptide is encoded by a nucleic acid at least 80% identical to SEQ ID NO:4 or a fragment thereof. In some embodiments, the IL-2 peptide comprises an amino acid sequence at least 80% identical to SEQ ID NO:13 or 18 or a fragment thereof. In some embodiments, the interleukin-2 (IL-2) peptide is encoded by SEQ ID NO:4.

In some embodiments, IgG1 peptide is encoded by a nucleic acid at least 80% identical to SEQ ID NO:7 or a fragment thereof. In some embodiments, the IgG1 peptide comprises an amino acid sequence at least 80% identical to SEQ ID NO:14 or 16 or a fragment thereof. In some embodiments, the IgG1 peptide is encoded by SEQ ID NO:7. In some embodiments, the IgG1 peptide comprises an amino acid sequence identical to SEQ ID NO: 14 or 16.

In some embodiments, the TL1A peptide is encoded by a nucleic acid at least 80% identical to SEQ ID NO:9 or a fragment thereof. In some embodiments, the TL1A peptide comprises an amino acid sequence at least 80% identical to SEQ ID NO:15 or 17 or a fragment thereof. In some embodiments, the TL1A peptide is encoded by SEQ ID NO:9. In some embodiments, the TL1A peptide comprises an amino acid sequence identical to SEQ ID NO: 15 or 17.

In some embodiments, the chimeric polypeptide is encoded by a nucleic acid at least 80% identical to SEQ ID NO:11 or a fragment thereof. In some embodiments, the chimeric polypeptide comprises an amino acid sequence at least 80% identical to SEQ ID NO:12 or a fragment thereof. In some embodiments, the chimeric polypeptide is encoded by SEQ ID NO: 11.

In some aspects, disclosed herein is a pharmaceutical composition comprising: a chimeric polypeptide comprising:
an interleukin-2 (IL-2) peptide;
an IgG1 peptide; and
a TL1A peptide;
and a pharmaceutically acceptable carrier.

In some aspects, disclosed herein is a method of preventing graft rejection in a subject, comprising administering to the subject a therapeutically effective amount of a chimeric polypeptide comprising:
an interleukin-2 (IL-2) peptide;
an IgG1 peptide; and
a TL1A peptide.

In some aspects, disclosed herein is a method of preventing graft vs. host disease (GVHD) in a subject, comprising administering to the subject a therapeutically effective amount of a chimeric polypeptide comprising:
an interleukin-2 (IL-2) peptide;
an IgG1 peptide; and
a TL1A peptide.

In some aspects, disclosed herein is a method of treating or suppressing an autoimmune disease in a subject, comprising administering to the subject a therapeutically effective amount of a chimeric polypeptide comprising:
an interleukin-2 (IL-2) peptide;
an IgG1 peptide; and
a TL1A peptide.

In some embodiments, the autoimmune disease is selected from Type 1 Diabetes (TID), inflammatory bowel disease (IBD), systemic lupus erythematosus (SLE), and rheumatoid arthritis (RA).

### BRIEF DESCRIPTION OF THE DRAWINGS

The accompanying figures, which are incorporated in and constitute a part of this specification, illustrate several aspects described below.

FIG. 1 shows schematic cartoon of the fusion protein. The IL-2 protein is attached via a linker to the CH2/CH3 domains of murine IgG1. The leader sequence (LS) is not shown in the protein model since it is cleaved after export.

FIG. 2 shows test for IL-2 function of the IL2-IgG1-TL1A FP indicates the FP stimulates IL-2 dependent CTLL cell proliferation via it's IL-2 moiety. Supernatant from CHO-K1 transfected cells 48 hrs. after transfection was tested using the IL-2 dependent CTLL cell line. An MTT assay was performed (duplicate wells) using CHO-K1 transfected supernatant. Together with results from the positive (rmIL-2) and negative (media alone) controls, the IL2-IgG1-TL1A FP clearly induced proliferation by the CTLL read-out cells.

FIG. 3 shows test for TL1A function of IL2-IgG1-TL1A FP shows the FP kills a P815 mouse tumor transfected with human TNFRSF25. CHO-K1 supernatant from transfected cells was collected after 48 hrs. of culture and titrated using duplicate wells containing the P815 huTNFRSF25 line. An MTT assay was performed to determine cell survival. Controls included wells containing no FP (medium only)—“positive control”, ie. cells survived indicating death of the P815 cells did not occur. The IL2-IgG1-TL1A FP clearly resulted in cell death through at least a 1:16 titration and killing diminished at higher dilutions of the supernatant.

FIGS. 4A-4B show function of IL2-IgG1-TL1A FP and comparison to TL1A-IgG1 FP plus free rhuIL-2. FIG. 4A) Culture supernatant (CS) was obtained from bulk cultures of transfected CHO-K1 cells and indicated volume injected i.p. in B6-FoxP3-RFP reporter mice on 4 consecutive days. Lymph node cells were assessed on day 7 for CD4 FoxP3⁺ expression by flow cytometric analysis. FIG. 4B) B6-FoxP3-RFP reporter mice were administered medium, purified TL1A-IgG1 FP (1 ug/inj; 2×/day) plus free rhuIL-2LD (2500 u/inj; 2×/day) or IL2-IgG1-TL1A FP (200 ul/inj=lug determined by ELISA) for 2 consecutive days. Lymph node cells were assessed on day 3 for CD4⁺ FoxP3⁺ expression. Recipients of IL2-IgG1-TL1A FP contained higher levels of lymph node CD4′FoxP3⁺ cells compared to recipients of TL1A-IgG1 FP plus free rhuIL-2LD.

### DETAILED DESCRIPTION

Disclosed herein is a novel in vivo strategy targeting two Treg receptors: TNFRSF25 and CD25. It was shown that combining TL1A-IgG1 administration together with IL-2 has a dramatic effect routinely raising overall Treg levels. The inventors have shown the effect of TNFRSF25+CD25 (IL2Ra) receptor stimulation on Tregs in hematopoietic stem cell transplants (HSCT) by manipulating donor Tregs and recipient Tregs in vivo.

The fusion proteins (FP) disclosed herein are a major advance and combine both TL1A and IL-2. Such a fusion protein can prolong the half-life of IL-2 and may allow lower TL1A levels. Thus, the inventors generated an IL-2-IgG1-TL1A fusion protein and tested its efficacy in vitro and in vivo. In vitro findings demonstrated that both the TL1A and IL-2 FP subunits were functional. It was next found that in vivo administration of very low levels of the fusion proteins disclosed herein increased Treg frequency, even at two days post-injections.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

The following definitions are provided for the full understanding of terms used in this specification.

### Terminology

As used herein, the article “a,” “an,” and “the” means “at least one,” unless the context in which the article is used clearly indicates otherwise.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed herein.

The term “nucleic acid” as used herein means a polymer composed of nucleotides, e.g., deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin; for example, promoters derived from viruses or from other organisms can be used in the compositions, systems, or methods described herein. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cells and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol I promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the β-actin promoter, the phosphoglycerol kinase (PGK) promoter, and the EFla promoter. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981). It is appreciated by those skilled in the art that the design of the expression vector can depend on such factors as the choice of the host cell to be transformed, the level of expression desired, etc.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “recombinant” or “chimeric” refers to a human manipulated nucleic acid (e.g., polynucleotide) or a copy or complement of a human manipulated nucleic acid (e.g., polynucleotide), or if in reference to a protein (i.e., a “recombinant protein” or “chimeric protein”), a protein encoded by a recombinant nucleic acid or chimeric nucleic acid (e.g., polynucleotide). In embodiments, a recombinant expression cassette comprising a promoter operably linked to a second nucleic acid (e.g., polynucleotide) may include a promoter that is heterologous to the second nucleic acid (e.g., polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g., polynucleotides) combined in such a way that the nucleic acids (e.g., polynucleotides) are extremely unlikely to be found in nature. For instance, human-manipulated restriction sites or plasmid vector sequences may flank or separate the promoter from the second nucleic acid (e.g., polynucleotide). One of skill will recognize that nucleic acids (e.g., polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g., polynucleotide) may include a promoter that is heterologous to the second nucleic acid (e.g., polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g., polynucleotide) may include a terminator that is heterologous to the second nucleic acid (e.g., polynucleotide) as the result of human manipulation. In some embodiments, the expression cassette comprises a promoter operably linked to a second nucleic acid (e.g., polynucleotide) and a terminator operably linked to the second nucleic acid (e.g., polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, for example 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P (N)), which provides an indication of the probability by which a match between two nucleotides or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g., enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g., modulating relative to the absence of the promoter) the expression of a protein from that coding sequence (i.e., the coding sequence is under the transcriptional control of the promoter).

As used throughout, by a “subject” (or a “host”) is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human.

The term “about” as used herein when referring to a measurable value such as an amount, a percentage, and the like, is meant to encompass variations of +20%, +10%, +5%, or +1% from the measurable value.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level so long as the increase is statistically significant.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level so long as the decrease is statistically significant.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, palliatively or remedially.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, cells, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active cells, salts, esters, amides, proagents, conjugates, active metabolites, isomers, fragments, analogs, etc.

“Therapeutically effective amount” or “therapeutically effective dose” of a composition (e.g., a composition comprising an agent) refers to an amount that is effective to achieve a desired therapeutic result. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

### Polypeptides, Compositions, and Methods

In some aspects, disclosed herein is a chimeric polypeptide comprising:
an interleukin-2 (IL-2) peptide;
an immunoglobulin peptide; and
a TL1A peptide.

In some embodiments, the polypeptide further comprises a peptide linker between the IL-2 peptide and the immunoglobulin peptide. In some embodiments, the peptide linker is a 20 amino acid peptide linker. In some embodiments, the peptide linker is encoded by SEQ ID NO:5. Other linkers can also be employed, for example, including any (G3S)ₓ and (G4S)ₓ sequences. In some embodiments, the linker is about 10, 11, 12, 13, 14, 15, 16, 17, 18, 20, 22, 24, 26, 28, or 30 amino acids in length. In some embodiments, the linker is about 12 amino acids in length.

In some embodiments, the interleukin-2 (IL-2) peptide is encoded by a nucleic acid at least 80% (for example, at least 80%, 85%, 90%, 95%, 98%, or 99%) identical to SEQ ID NO:4 or a fragment thereof. In some embodiments, the interleukin-2 (IL-2) peptide is encoded by SEQ ID NO: 4 or a fragment thereof. In some embodiments, the IL-2 peptide comprises an amino acid sequence at least 80% (for example, at least 80%, 85%, 90%, 95%, 98%, or 99%) identical to SEQ ID NO: 13 or 18 or a fragment thereof. In some embodiments, the IL-2 peptide comprises SEQ ID NO: 13 or 18 or a fragment thereof.

In some embodiments, the immunoglobulin peptide is an IgG1 peptide, an IgG2 peptide, an IgG3 peptide, an IgG4 peptide, or an IgA peptide. In some embodiments, the immunoglobulin peptide is a IgG1 peptide. The immunoglobulin peptide used herein can pass blood brain barrier. Accordingly, in some aspects, disclosed herein is a chimeric polypeptide comprising:
an interleukin-2 (IL-2) peptide;
an IgG1 peptide; and
a TL1A peptide.

In some embodiments, the IgG1 peptide is encoded by a nucleic acid at least 80% (for example, at least 80%, 85%, 90%, 95%, 98%, or 99%) identical to SEQ ID NO:7 or a fragment thereof. In some embodiments, the IgG1 peptide is encoded by SEQ ID NO:7. In some embodiments, the IgG1 peptide comprises an amino acid sequence at least 80% (for example, at least 80%, 85%, 90%, 95%, 98%, or 99%) identical to SEQ ID NO: 14 or 16 or a fragment thereof. In some embodiments, the IgG1 peptide comprises SEQ ID NO: 14 or 16 or a fragment thereof.

In some embodiments, the TL1A peptide is encoded by a nucleic acid at least 80% (for example, at least 80%, 85%, 90%, 95%, 98%, or 99%) identical to SEQ ID NO:9 or a fragment thereof. In some embodiments, the TL1A peptide is encoded by SEQ ID NO:9. In some embodiments, the TL1A peptide comprises an amino acid sequence at least 80% (for example, at least 80%, 85%, 90%, 95%, 98%, or 99%) identical to SEQ ID NO:15 or 17 or a fragment thereof. In some embodiments, the TL1A peptide comprises SEQ ID NO: 15 or 17 or a fragment thereof.

In some embodiments, the chimeric polypeptide or recombinant polypeptide is encoded by a nucleic acid at least 80% identical to SEQ ID NO:11. In some embodiments, the chimeric polypeptide or recombinant polypeptide is encoded by SEQ ID NO: 11. In some embodiments, the chimeric polypeptide or recombinant polypeptide comprises an amino acid sequence at least 80% identical to SEQ ID NO:12 or a fragment thereof. In some embodiments, the chimeric polypeptide or recombinant polypeptide comprises SEQ ID NO: 12.

In some embodiments, the interleukin-2 (IL-2) peptide is a mouse IL-2 peptide. In some embodiments, the interleukin-2 (IL-2) peptide is a human IL-2 peptide.

In some embodiments, the TL1A peptide is a mouse TL1A peptide. In some embodiments, the TL1A peptide is a human TL1A peptide.

In some embodiments, the IgG1 peptide is a mouse IgG1 peptide. In some embodiments, the IgG1 peptide is a human IgG1 peptide.

It should be understood and herein completed that the fusion protein or recombinant protein disclosed herein can prolong the half-life of an IL-2 peptide (e.g., at least an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level).

Also disclosed herein is a polynucleotide encoding the polypeptide or fusion protein disclosed herein. In some aspect, disclosed herein is a vector comprising the polynucleotide disclosed herein.

In some aspects, disclosed herein is a pharmaceutical composition comprising:

a chimeric polypeptide comprising:
an interleukin-2 (IL-2) peptide;
an immunoglobulin peptide (an IgG1 peptide, an IgG2 peptide, an IgG3 peptide, an IgG4
peptide, or an IgA peptide); and
a TL1A peptide;
and a pharmaceutically acceptable carrier.

In some embodiments, the immunoglobulin peptide is an IgG1 peptide.

In some aspects, disclosed herein is a method of preventing graft rejection in a subject, comprising administering to the subject a therapeutically effective amount of a chimeric polypeptide comprising:
an interleukin-2 (IL-2) peptide;
an immunoglobulin peptide (an IgG1 peptide, an IgG2 peptide, an IgG3 peptide, an IgG4
peptide, or an IgA peptide); and
a TL1A peptide.

In some embodiments, the immunoglobulin peptide is an IgG1 peptide.

In some aspects, disclosed herein is a method of preventing transplant rejection in a subject, comprising administering to the subject a therapeutically effective amount of a chimeric polypeptide comprising:
an interleukin-2 (IL-2) peptide;
an immunoglobulin peptide (an IgG1 peptide, an IgG2 peptide, an IgG3 peptide, an IgG4
peptide, or an IgA peptide); and
a TL1A peptide.

In some embodiments, the immunoglobulin peptide is an IgG1 peptide.

In some aspects, disclosed herein is a method of preventing graft vs. host disease (GVHD) in a subject, comprising administering to the subject a therapeutically effective amount of a chimeric polypeptide comprising:
an interleukin-2 (IL-2) peptide;
an immunoglobulin peptide (an IgG1 peptide, an IgG2 peptide, an IgG3 peptide, an IgG4
peptide, or an IgA peptide); and
a TL1A peptide.

In some embodiments, the immunoglobulin peptide is an IgG1 peptide.

Any of the IL-2 and TL1A chimeric polypeptides and nucleic acids disclosed herein can be used in the methods described above.

As used herein, the term “graft-versus-host disease” or “GVHD” refers to a condition, including acute and chronic, resulting from transplanted (graft) cell effects on host cells and tissues resulting from an allogeneic hematopoietic cell transplant. In other words, donor immune cells infused within the graft or donor immune cells that develop from the stem cells, may see the patient's (host) cells as foreign and turn against them with an immune response. As examples, patients who have had a blood or marrow transplant from someone else are at risk of having acute GVHD. Even donors who are HLA-matched with the recipient can cause GVHD because the donor cells can potentially also make an immune response against minor antigen differences in the recipient. Acute graft-versus-host disease (GVHD) is a disorder caused by donor immune cells in patients who have had an allogeneic marrow or blood cell transplantation. The most commonly affected tissues are skin, intestine and liver. In severe cases, GVHD can cause blistering in the skin or excessive diarrhea and wasting. Also, inflammation caused by donor immune cells in the liver can cause obstruction that causes jaundice. Other tissues such as lung and thymus may also become affected. The diagnosis is usually confirmed by looking at a small piece of skin, liver, stomach or intestine with a microscope for observation of specific inflammatory characteristics. The symptoms of acute GVHD further comprises an increase of white blood cell counts and proinflammatory cytokine levels. The symptoms of acute GVHD usually begins within the first 3 months after the transplant. In some cases, it can persist, come back or begin more than 3 months after the transplant. In some examples, the compositions and methods disclosed herein are used for treatment and/or prevention of acute GVHD.

The compositions and methods disclosed herein for preventing and/or treating acute GVHD can be a prevention and/or treatment of one or more of blistering in the skin, skin rashes, abdominal cramps, excessive diarrhea, inflammation in the liver, intestine, lung, thymus, jaundice, and/or nausea. In some embodiments, the administration of the composition disclosed herein increases levels of Treg and/or IL-10 (for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level). In some embodiments, the administration of the composition disclosed herein decreases levels of proinflammatory cytokines and/or white blood cell counts (e.g., a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease).

In some embodiments, the method of any preceding aspect further comprises administering to the subject a therapeutically effective amount of an additional agent for treating GVHD. In some embodiments, the additional agent is selected from the group consisting of methotrexate, cyclosporine, tacrolimus, mycophenolate mofetil, sirolimus, corticosteroid, anti-thymocyte globulin, alemtuzumab, and cyclophosphamide.

In some aspects, disclosed herein is a method of treating, preventing, or suppressing an autoimmune disease in a subject, comprising administering to the subject a therapeutically effective amount of a chimeric polypeptide comprising:
an interleukin-2 (IL-2) peptide;
an immunoglobulin peptide (an IgG1 peptide, an IgG2 peptide, an IgG3 peptide, an IgG4
peptide, or an IgA peptide); and
a TL1A peptide.

In some embodiments, the immunoglobulin peptide is an IgG1 peptide

The term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia greata, ankylosing spondylitis, autoimmune hepatitis, autoimmune parotitis, Crohn's disease, diabetes (Type 1), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjögren's syndrome, spondyloarthropathies, thyroiditis, vasculitis, vitiligo, myxedema, pernicious anemia, ulcerative colitis, among others.

In some embodiments, the autoimmune disease is selected from Type 1 Diabetes (TID), inflammatory bowel disease (IBD), systemic lupus erythematosus (SLE), and rheumatoid arthritis (RA).

In some aspects, disclosed herein is a method of modulating an immune response in a subject, comprising administering to the subject a chimeric polypeptide comprising:
an interleukin-2 (IL-2) peptide;
an immunoglobulin peptide (an IgG1 peptide, an IgG2 peptide, an IgG3 peptide, an IgG4
peptide, or an IgA peptide); and
a TL1A peptide.

In some embodiments, the immunoglobulin peptide is an IgG1 peptide.

In some aspects, disclosed herein is a method of strengthening or repairing Treg cells (CD4′FoxP3⁺) in a subject, comprising administering to the subject a chimeric polypeptide comprising:
an interleukin-2 (IL-2) peptide;
an immunoglobulin peptide (an IgG1 peptide, an IgG2 peptide, an IgG3 peptide, an IgG4
peptide, or an IgA peptide); and
a TL1A peptide.

In some embodiments, the immunoglobulin peptide is an IgG1 peptide.

In some aspects, disclosed herein is a method for in vivo expansion of Treg cells in a subject, comprising administering to the subject a chimeric polypeptide comprising:
an interleukin-2 (IL-2) peptide;
an immunoglobulin peptide (an IgG1 peptide, an IgG2 peptide, an IgG3 peptide, an IgG4
peptide, or an IgA peptide); and
a TL1A peptide.

In some embodiments, the immunoglobulin peptide is an IgG1 peptide

Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, intravaginal, by inhalation, via an implanted reservoir, or via a transdermal patch, and the like.

Dosing frequency for the composition disclosed herein, includes, but is not limited to, at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks, once a week, twice a week, three times a week, four times a week, five times a week, six times a week, or daily. In some embodiments, dosing frequency for the composition disclosed herein includes at least once every 12 months, once every 11 months, once every 10 months, once every 9 months, once every 8 months, once every 7 months, once every 6 months, once every 5 months, once every 4 months, once every 3 months, once every two months, once every month; or at least once every three weeks, once every two weeks or once a week. In some embodiments, the interval between each administration is less than about 4 months, less than about 3 months, less than about 2 months, less than about a month, less than about 3 weeks, less than about 2 weeks, or less than less than about a week, such as less than about any of 6, 5, 4, 3, 2, or 1 day. In some embodiments, the dosing frequency for genetically modified cell includes, but is not limited to, at least once a day, twice a day, or three times a day. In some embodiments, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiments, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiments, the interval between each administration is less than about 4 months, less than about 3 months, less than about 2 months, less than about a month, less than about 3 weeks, less than about 2 weeks, or less than less than about a week. In some embodiments, the interval between each administration is constant. For example, the administration can be carried out daily, every two days, every three days, every four days, every five days, or weekly. In some examples, the administration can be carried out every week, every two weeks, or every two months

### EXAMPLES

The following examples are set forth below to illustrate the chimeric polypeptides, compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. TL1A and IL-2 Fusion Proteins

Tregs are a non-redundant CD4⁺ T cell population required for immune homeostasis/maintenance of peripheral tolerance. These cells suppress immune responses and inflammation and promote tissue repair, with clinical application to prevent liquid and solid organ graft rejection. Two major strategies to exploit Treg function are: 1) ex vivo expansion and subsequent cell therapy, or 2) in vivo expansion to increase numbers/function. Administration of low dose IL-2 can augment Tregs in situ and this approach is being tested in transplant and autoimmune studies. Importantly, ex vivo expansion is limited to Tregs from blood, whereas in vivo expansion can directly impact Tregs in target/diseased compartments, thus obviating trafficking requirements.

Disclosed herein is a novel in vivo strategy targeting two Treg receptors: TNFRSF25 and CD25. It was shown that targeting TNFRSF25 in vivo with an agonistic TL1A-IgG1 fusion protein (FP) (TNFRSF25 natural ligand and mouse IgG1) expanded Tregs to a greater level (˜2-3×) vs. IL-2 alone. It was also shown that combining FP administration together with IL-2 has a dramatic and transient effect routinely raising overall Treg levels (40-70%). It has been shown that the effect of TNFRSF25 (TL1A Receptor)+CD25 (IL2Ra) stimulation on Tregs in hematopoietic stem cell transplants (HSCT) by manipulating donor Tregs and recipient Tregs in vivo.

The fusion proteins (FP) disclosed herein are a major advance and combine both TL1A and IL-2. Such a fusion protein can prolong the half-life of IL-2, can require lower TL1A levels and more readily enable use of multiple infusions. Thus, the inventors generated an IL-2-IgG1-TL1A fusion protein and tested its efficacy in vitro and in vivo (mice). In vitro findings demonstrated that both the TL1A and IL-2 FP subunits were functional. It was next found that in vivo administration of very low levels of FP increased Treg frequency, even two days post-injections.

There continues to be a need for successful long-term transplantation (permanent graft acceptance) of both liquid (for example, bone marrow, islet cells) and solid organ (for example, liver, heart) transplantation because 50% of organs are rejected within 10-12 years, and 30-70% of bone marrow transplants result in acute or chronic GVHD. Controlling the immune system to prevent destruction of self-tissues and cells (autoimmune disease) remains an enormous challenge.

Both conditions require long-term immunosuppression (IS) and current IS treatments are largely global and non-specific, resulting in major off-target/unwanted side effects. Novel targeted therapies represent a major clinical advance for these patients.

Autoimmune diseases including TID, IBD, SLE, RA (there are >70 diseases) are affecting ˜8% of Americans (˜25,000,000 individuals in the US). Greater than 60,000 liquid+ solid transplants are performed annually in the US which would increase significantly if graft rejection could be controlled and ultimately prevented.

Immunosuppressive and anti-inflammatory compounds are currently primarily prescribed to transplant (and autoimmune disease) patients. Immunosuppressive drugs including cyclosporine, rapamycin, methotrexate and others have significant/varying off-target effects. Anti-inflammatory compounds (for example, corticosteroids, prednisone, dexamethasone) are broadly immunosuppressive with major side-affects. The use of anti-T cell depleting antibodies is employed to inhibit transplant rejection; rituximab (anti-CD20 mAB) is used for patients with autoimmune disease (for example, lupus, Graves's).

Other therapies involve the isolation and purification of Tregs from the peripheral blood of individuals and ex vivo expansion using beads and cytokines for subsequent inoculation into patients with GVHD and Type I diabetes. CAR-Tregs are also under development.

Disclosed herein is a fusion protein (FP) composed of murine IL-2, a 20 amino acid linker plus IgG1-TL1A.

These fusion proteins circumvent the need to expand Tregs ex vivo by expanding these cells in vivo to prevent graft rejection and suppress autoimmune disease.

Drugs including anti-inflammatoires mediate many side effects and target multiple genes in all cells. Additionally, mAbs have a longer half-life versus fusion proteins (FP). The FP herein selectively stimulate the patient's immune system to inhibit unwanted immune responses underlying graft rejection and autoimmune disorders.

The FP herein targets two receptors on Treg cells (TNFRSF25 and CD25) and is more potent compared to free rIL-2 alone.

In addition, the fusion proteins herein can act as a carrier to deliver a “cytotoxic payload” —thereby generating a strategy to eliminate Treg cells with applications directed to augmenting immune responses (for example, for vaccines or cancer).

### Example 2. Generation of IL2-(G4S) 4 Linker-IgG1-TL1A (IL2-IgG1-TL1A FP)

A fusion protein (FP) was generated comprised of murine IL-2, a 20 amino acidlinker, mouse IgG1, and TL1A—the physiological ligand for TNFRSF25 (FIG. 1).

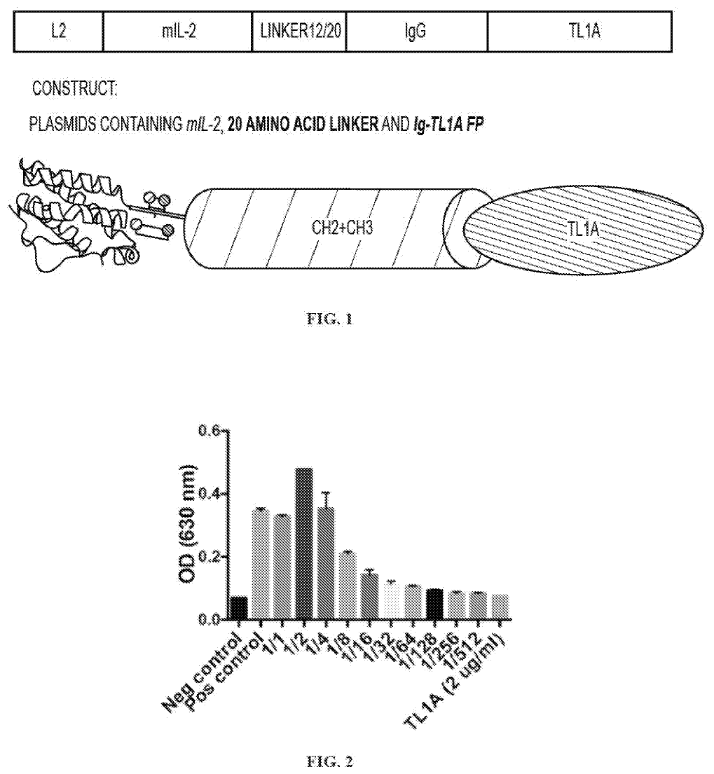

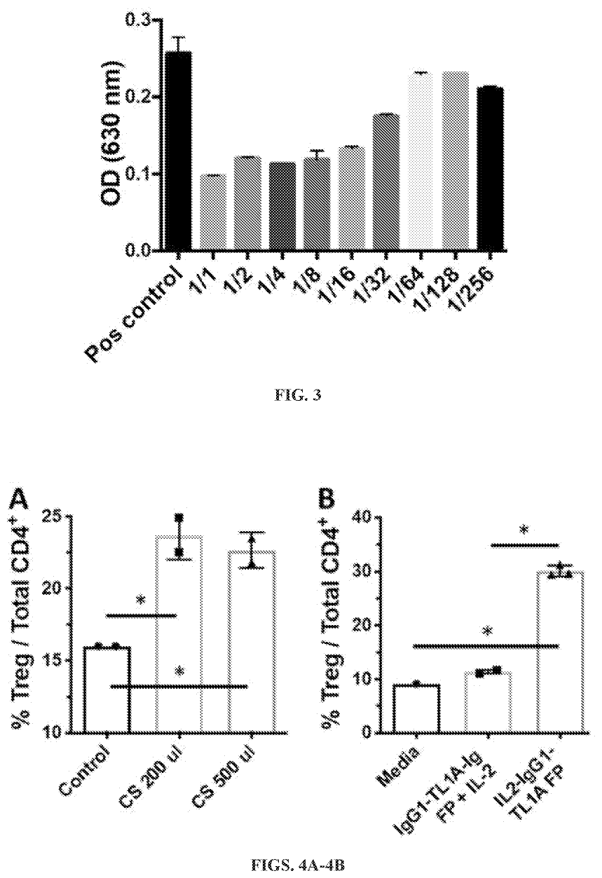

To begin testing the FP for functional activity, supernatant from CHO-K1 transfected cells was collected after 48 hrs. of culture and titrated using duplicate wells containing the IL-2 dependent CTLL cell line. Controls included recombinant mouse IL-2 (20 u/ml=“positive”) and no IL-2, media (“negative”). After 48 hours, proliferation was assessed (FIG. 2). Cell growth induced by the FP was equivalent to the positive control through a 1:4 FP dilution. Importantly, a TL1A-IgG1 FP was also included to determine if the proliferation could have been induced via the TL1A protein on the IL2-IgG1-TL1A FP. The absence of proliferation conclusively determined that the IL-2 moiety of the IL2-IgG1-TL1A FP was functional (FIG. 2). The FP was next analyzed to assess TL1A function using a read-out cell line (mouse P815) transfected to express human TNFRSF25. Prior studies showed that high expression of TNFRSF25 on this cell could signal apoptotic cell death via the death domain on the intracytoplasmic domain of TNFRSF25 (Khan, 2013). Titration of the same IL2-IgG1-TL1A FP containing supernatant used to demonstrate IL-2 function (FIG. 2) also demonstrated TL1A function as assessed by loss of P815 cells in the MTT assay through a 1:16 dilution (FIG. 3). Microscopic photos confirmed the MTT results (data not shown).

It was reported that Treg cells expressed TNFRSF25 and agonistic stimulation provided in vivo could expand CD4⁺ FoxP3⁺ Tregs to significantly higher levels compared to IL-2 (Schreiber, 2010 #2; Khan, 2013 #1, Wolf, 2017 #3). It was also discovered that combined administration of TL1A-IgG1 FP and free rhuIL-2 resulted in marked expansion superior to either reagent individually (Wolf, 2017 #3, 2018 #4).

Following in vitro evaluation of the fusion proteins herein, IL2-IgG1-TL1A FP, in vivo function of this FP was analyzed as whether the FP could expand Tregs in vivo and whether expansion was superior to combined TL1A-IgG1 plus free rhuIL-2 (FIG. 4). It was observed that culture supernatant from bulk IL2-IgG1-TL1A transfected cells induced significant (˜70-80% increase) Treg expansion (FIG. 4A). Next, the original TL1A-IgG1 FP together with free rhuIL-2 was compared to the novel FP herein containing IL2-IgG1-TL1A.

Administering comparable amounts of reagents, after only 2 days of injections, the IL2-IgG1-TL1A FP induced an ˜3× expansion of Tregs. Notably, there was marginal if any expansion using the individual TL1A-IgG1 FP and free rhuIL-2 (FIG. 4B).

### REFERENCES

1. Khan S Q, Tsai M S, Schreiber T H, Wolf D, Deyev V V, Podack E R. Cloning, expression, and functional characterization of TL1A-Ig. J Immunol. 2013 Feb. 15;190 (4): 1540-50. doi: 10.4049/jimmunol. 1201908. Epub 2013 Jan. 14.
2. Schreiber T H, Wolf D, Tsai M S, Chirinos J, Deyev V V, Gonzalez L, Malek T R, Levy R B, Podack E R. Therapeutic Treg expansion in mice by TNFRSF25 prevents allergic lung inflammation.J Clin Invest. 2010 October;120 (10): 3629-40. doi: 10.1172/JCI42933. Epub 2010 Sep. 20.
3. Wolf D, Barreras H, Bader C S, Copsel S, Lightbourn C O, Pfeiffer B J, Altman N H, PodackER, Komanduri K V, Levy R B. Marked in Vivo Donor Regulatory T Cell Expansion via Interleukin-2 and TL1A-Ig Stimulation Ameliorates Graft-versus-Host Disease but Preserves Graft-versus-Leukemia in Recipients afterHematopoietic Stem Cell Transplantation.

Biol Blood Marrow Transplant. 2017 May;23 (5): 757-766. doi: 10.1016/j.bbmt.2017.02.013. Epub2017 February 20.
4. Wolf D, Bader C S, Barreras H, Copsel S, Pfeiffer B J, Lightbourn C O, Altman N H, KomanduriKV, Levy R B. Superior immune reconstitution using Treg-expanded donor cells versus PTCy treatment in preclinical HSCT models. JCI Insight. 2018 Oct. 18;3 (20): e121717. doi: 10.1172/jci.insight. 121717.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

```
SEQUENCES
mIL2_{20 AA linker}-mlgG1-mTL1A sequence
  (SEQ ID NO: 1)
gtcgac (SalI site) gccacc (Kozak seq.)
  (SEQ ID NO: 2)
atggagacagacacactcctgctatgggtactgctgctctgggttccaggttccactggtgac (leader seq)
  (SEQ ID NO: 3)
ctcgag (XhoI site) (IL-2 start)
  (SEQ ID NO: 4)
gccaccatggacagcatgcagctcgcatcctgtgtcacattgacacttgtgctccttgtcaacagcgcacccacttcaagctctacttcaag
ctctacagcggaagcacagcagcagcagcagcagcagcagcagcagcagcacctggagcagctgttgatggacctacaggagctcctga
gcaggatggagaattacaggaacctgaaactccccaggatgctcaccttcaaattttacttgcccaagcaggccacagaattgaaagatctt
cagtgcctagaagatgaacttggacctctgcggcatgttctggatttgactcaaagcaaaagctttcaattggaagatgctgagaatttcat
cagcaatatcagagtaactgttgtaaaactaaagggctctgacaacacatttgagtgccaattcgatgatgagtcagcaactgtggtggact
ttctgaggagatggatagccttctgtcaaagcatcatctcaacaagccctcaa (IL-2 end)
  (SEQ ID NO: 5)
ggaggcgggggttccggaggcgggggttcaggaggcgggggttccggaggcgggggttca (20 AA linker)
  (SEQ ID NO: 6)
ctcgag (XhoI site) (start mIgG1)
  (SEQ ID NO: 7)
gtgcccagggattctggttctaagccttccatatctacagtcccagaagtatcatctgtcttcatcttccccccaaagcccaaggatgtgct
caccattactctgactcctaaggtcacgtgtgttgtggtagacatcagcaaggatgatcccgaggtccagttcagctggtttgtagatgatg
tggaggtgcacacagctcagacaaaaccccgggaggagcagttcaacagcactttccgttcagtcagtgaacttcccatcatgcaccaggac
tggctcaatggcaaggagttcaaatgcagggtcaacagtgcagctttccctgcccccatcgagaaaaccatctccaaaaccaaaggcagacc
gaaggctccacaggtgtacaccattccacctcccaaggagcagatggccaaggataaagtcagtctgacctgcatgataacagacttcttcc
ctgaagacattactgtggagtggcagtggaatgggcagccagcggagaactacaagaacactcagcccatcatggacacagatggctcttac
ttcgtctacagcaagctcaatgtgcagaagagcaactgggaggcaggaaatactttcacctgctctgtgttacatgagggcctgcacaacca
ccatactgagaagagcctctcccactctcctggtaaa (end mIgG1)
  (SEQ ID NO: 8)
gaattc (EcoR1) (start mTL1A)
  (SEQ ID NO: 9)
cgggtccccggaaaagactgtatgcttcgggccataacagaagagagatctgagccttcaccacagcaagtttactcacctcccagaggca
agccgagagcacacctgacaattaagaaacaaaccccagcaccacatctgaaaaatcagctctctgctctacactgggaacatgacctagg
gatggccttcaccaagaacgggatgaagtacatcaacaaatccctggtgatcccagagtcaggagactatttcatctactcccagatcacat
tccgagggaccacatctgtgtgtggtgacatcagtcgggggagacgaccaaacaagccagactccatcaccatggttatcaccaaggtagc
agacagctaccctgagcctgcccgcctactaacagggtccaagtctgtgtgtgaaataagcaacaactggttccagtccctctaccttgggg
ccacgttctccttggaagaaggagacagactaatggtaaacgtcagtgacatctccttggtggattacacaaaagaagataaaactttcttt
ggagctttcttgctataa (end TL1A + stop)
  (SEQ ID NO: 10)
gcggccgc (NotI site)
Translation starts at the Xho site:
DNA = (SEQ ID NO: 11)
ctcgaggccaccatggacagcatgcagctcgcatcctgtgtcacattgacacttgtgctccttgtcaacagcgcacccacttcaagctctact
tcaagctctacagcggaagcacagcagcagcagcagcagcagcagcagcagcagcacctggagcagctgttgatggacctacaggagc
tcctgagcaggatggagaattacaggaacctgaaactccccaggatgctcaccttcaaattttacttgcccaagcaggccacagaattgaaag
atcttcagtgcctagaagatgaacttggacctctgcggcatgttctggatttgactcaaagcaaaagctttcaattggaagatgctgagaatt
tcatcagcaatatcagagtaactgttgtaaaactaaagggctctgacaacacatttgagtgccaattcgatgatgagtcagcaactgtggtgg
actttctgaggagatggatagccttctgtcaaagcatcatctcaacaagccctcaaggaggcgggggttccggaggcgggggttcaggagg
cgggggttccggaggcgggggttcactcgaggtgcccagggattctggttctaagccttccatatctacagtcccagaagtatcatctgtctt
catcttccccccaaagcccaaggatgtgctcaccattactctgactcctaaggtcacgtgtgttgtggtagacatcagcaaggatgatcccga
ggtccagttcagctggtttgtagatgatgtggaggtgcacacagctcagacaaaaccccgggaggagcagttcaacagcactttccgttca
gtcagtgaacttcccatcatgcaccaggactggctcaatggcaaggagttcaaatgcagggtcaacagtgcagctttccctgcccccatcga
gaaaaccatctccaaaaccaaaggcagaccgaaggctccacaggtgtacaccattccacctcccaaggagcagatggccaaggataaag
tcagtctgacctgcatgataacagacttcttccctgaagacattactgtggagtggcagtggaatgggcagccagcggagaactacaagaa
cactcagcccatcatggacacagatggctcttacttcgtctacagcaagctcaatgtgcagaagagcaactgggaggcaggaaatactttca
cctgctctgtgttacatgagggcctgcacaaccaccatactgagaagagcctctcccactctcctggtaaagaattccgggtccccggaaaa
gactgtatgcttcgggccataacagaagagagatctgagccttcaccacagcaagtttactcacctcccagaggcaagccgagagcacac
ctgacaattaagaaacaaaccccagcaccacatctgaaaaatcagctctctgctctacactgggaacatgacctagggatggccttcaccaa
gaacgggatgaagtacatcaacaaatccctggtgatcccagagtcaggagactatttcatctactcccagatcacattccgagggaccacat
ctgtgtgtggtgacatcagtcgggggagacgaccaaacaagccagactccatcaccatggttatcaccaaggtagcagacagctaccctgag
cctgcccgcctactaacagggtccaagtctgtgtgtgaaataagcaacaactggttccagtccctctaccttggggccacgttctccttggaa
gaaggagacagactaatggtaaacgtcagtgacatctccttggtggattacacaaaagaagataaaactttctttggagctttcttgctataa
Protein = (SEQ ID NO: 12)
LEATMDSMQLASCVTLTLVLLVNSAPTSSSTSSSTAEAQQQQQQQQQQQHLEQLLMDL
QELLSRMENYRNLKLPRMLTFKFYLPKQATELKDLQCLEDELGPLRHVLDLTQSKSFQL
EDAENFISNIRVTVVKLKGSDNTFECQFDDESATVVDFLRRWIAFCQSIISTSPQGGGGSG
GGGSGGGGSGGGGSLEVPRDSGSKPSISTVPEVSSVFIFPPKPKDVLTITLTPKVTCVVVD
ISKDDPEVQFSWFVDDVEVHTAQTKPREEQFNSTFRSVSELPIMHQDWLNGKEFKCRVN
SAAFPAPIEKTISKTKGRPKAPQVYTIPPPKEQMAKDKVSLTCMITDFFPEDITVEWQWN
GQPAENYKNTQPIMDTDGSYFVYSKLNVQKSNWEAGNTFTCSVLHEGLHNHHTEKSLS
HSPGKEFRVPGKDCMLRAITEERSEPSPQQVYSPPRGKPRAHLTIKKQTPAPHLKNQLSA
LHWEHDLGMAFTKNGMKYINKSLVIPESGDYFIYSQITFRGTTSVCGDISRGRRPNKPDS
ITMVITKVADSYPEPARLLTGSKSVCEISNNWFQSLYLGATFSLEEGDRLMVNVSDISLV
DYTKEDKTFFGAFLL
Protein sequence for individual parts (restriction sites and linker not included)
mIL-2:
  (SEQ ID NO: 13)
ATMDSMQLASCVTLTLVLLVNSAPTSSSTSSSTAEAQQQQQQQQQQQHLEQLLMDLQE
LLSRMENYRNLKLPRMLTFKFYLPKQATELKDLQCLEDELGPLRHVLDLTQSKSFQLED
AENFISNIRVTVVKLKGSDNTFECQFDDESATVVDFLRRWIAFCQSIISTSPQ
mIgG1:
  (SEQ ID NO: 14)
VPRDSGSKPSISTVPEVSSVFIFPPKPKDVLTITLTPKVTCVVVDISKDDPEVQFSWFVDD
VEVHTAQTKPREEQFNSTFRSVSELPIMHQDWLNGKEFKCRVNSAAFPAPIEKTISKTKG
RPKAPQVYTIPPPKEQMAKDKVSLTCMITDFFPEDITVEWQW.NGQPAENYKNTQPIMD
TDGSYFVYSKLNVQKSNWEAGNTFTCSVLHEGLHNHHTEKSLSHSPGK
mTL1A:
  (SEQ ID NO: 15)
RVPGKDCMLRAITEERSEPSPQQVYSPPRGKPRAHLTIKKQTPAPHLKNQLSALHWEHD
LGMAFTKNGMKYINKSLVIPESGDYFIYSQITFRGTTSVCGDISRGRRPNKPDSITMVITK
VADSYPEPARLLTGSKSVCEISNNWFQSLYLGATFSLEEGDRLMVNVSDISLVDYTKED
KTFFGAFLL
Human counterparts to mouse sequences
Human IgG1 (hinge + C2 + C3; 229 aa):
  (SEQ ID NO: 16)
CDKTHTCPPCPAPELLGGPSVFLFPPKPKDTLISRTPEVTCVVVDVSHEDPEVKFNWYVD
GVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
Human TL1A (full extracell. Domain; 191 aa):
  (SEQ ID NO: 17)
EFRAQGEACVQFQA(cleavagesite)LKGQEFAPSHQQVYAPLRADGDKPRAHLTVVRQTP
TQHFKNQFPALHWEHELGLAFTKNRMNYTNKFLLIPESGDYFIYSQVTFRGMTSECSEIR
QAGRPNKPDSITVVITKVTDSYPEPTQLLMGTKSVCEVGSNWFQPIYLGAMFSLQEGDK
LMVNVSDISLVDYTKEDKTFFGAFLL
Human IL-2 (153 aa)
NM_000586.3 Homo sapiens interleukin 2 (IL2)
  (SEQ ID NO: 18)
MYRMQLLSCIALSLALVTNSAPTSSSTKKTQLQLEHLLLDLQMILNGINNYKNPKLTRM
LTFKFYMPKKATELKHLQCLEEELKPLEEVLNLAQSKNFHLRPRDLISNINVIVLELKGSE
TTFMCEYADETATIVEFLNRWITFCQSIISTLT
```

1. A chimeric polypeptide comprising:
an interleukin-2 (IL-2) peptide;
an immunoglobulin peptide; and
a TL1A peptide.

2. The polypeptide of claim 1, wherein the polypeptide further comprises a peptide linker between the IL-2 peptide and the immunoglobulin peptide.

3. The polypeptide of claim 2, wherein the peptide linker is a 20 amino acid peptide linker.

4. The polypeptide of claim 2, wherein the peptide linker is encoded by a nucleic acid at least 80% identical to SEQ ID NO:5 or a fragment thereof.

5. The polypeptide of claim 1, wherein the IL-2 peptide is encoded by a nucleic acid at least 80% identical to SEQ ID NO:4 or a fragment thereof.

6. The polypeptide of claim 1, wherein the IL-2 peptide comprises an amino acid sequence at least 80% identical to SEQ ID NO:13 or 18 or a fragment thereof.

7. The polypeptide of claim 1, wherein the immunoglobulin peptide is an IgG1 peptide.

8. The polypeptide of claim 7, wherein the IgG1 peptide is encoded by a nucleic acid at least 80% identical to SEQ ID NO:7 or a fragment thereof.

9. The polypeptide of claim 7, wherein the IgG1 peptide comprises an amino acid sequence at least 80% identical to SEQ ID NO:14 or 16 or a fragment thereof.

10. The polypeptide of claim 1, wherein the TL1A peptide is encoded by a nucleic acid at least 80% identical to SEQ ID NO:9 or a fragment thereof.

11. The polypeptide of claim 1, wherein the TL1A peptide comprises an amino acid sequence at least 80% identical to SEQ ID NO:15 or 17 or a fragment thereof.

12. The polypeptide of claim 1, wherein the chimeric polypeptide is encoded by a nucleic acid at least 80% identical to SEQ ID NO:11 or a fragment thereof.

13. The polypeptide of claim 1, wherein the chimeric polypeptide comprises an amino acid sequence at least 80% identical to SEQ ID NO:12 or a fragment thereof.

14. A polynucleotide encoding the polypeptide of claim 1.

15. A pharmaceutical composition comprising the chimeric polypeptide of claim 1 and a pharmaceutically acceptable carrier.

16. A pharmaceutical composition comprising the polynucleotide of claim 14 and a pharmaceutically acceptable carrier.

17. A method of preventing graft rejection in a subject, comprising administering to the subject a therapeutically effective amount of the chimeric polypeptide of claim 1.

18. A method of preventing graft vs. host disease (GVHD) in a subject, comprising administering to the subject a therapeutically effective amount of the chimeric polypeptide of claim 1.

19. A method of treating or suppressing an autoimmune disease in a subject, comprising administering to the subject a therapeutically effective amount of the chimeric polypeptide of claim 1.

20. The method of claim 19, wherein the autoimmune disease is selected from Type 1 Diabetes (TID), inflammatory bowel disease (IBD), systemic lupus erythematosus (SLE), and rheumatoid arthritis (RA).


SEQUENCE LISTING (18 sequences)

SEQ ID NO: 1 - DNA, 12 bp, synthetic construct
  misc_feature 1..12: note = Description of sequence: Kozak seq.
  misc_feature 1..12: note = Description of sequence: mIL220 AA linker-mIgG1-mTL1A sequence component
  source 1..12: mol_type = other DNA
gtcgacgccacc

SEQ ID NO: 2 - DNA, 63 bp, synthetic construct
  misc_feature 1..63: note = Description of sequence: leader seq
  misc_feature 1..63: note = Description of sequence: mIL220 AA linker-mIgG1-mTL1A sequence component
  source 1..63: mol_type = other DNA
atggagacagacacactcctgctatgggtactgctgctctgggttccaggttccactggtgac

SEQ ID NO: 3 - skipped

SEQ ID NO: 4 - DNA, 510 bp, synthetic construct
  misc_feature 1..510: note = Description of sequence: IL-2 end
  misc_feature 1..510: note = Description of sequence: mIL220 AA linker-mIgG1-mTL1A sequence component
  source 1..510: mol_type = other DNA
gccaccatggacagcatgcagctcgcatcctgtgtcacattgacacttgtgctccttgtcaacagcgcacccacttcaagctctacttcaagctctacagcggaagcacagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaa

SEQ ID NO: 5 - DNA, 60 bp, synthetic construct
  misc_feature 1..60: note = Description of sequence: 20 AA linker
  misc_feature 1..60: note = Description of sequence: mIL220 AA linker-mIgG1-mTL1A sequence component
  source 1..60: mol_type = other DNA
ggaggcgggggttccggaggcgggggttcaggaggcgggggttccggaggcgggggttca

SEQ ID NO: 6 - skipped

SEQ ID NO: 7 - DNA, 681 bp, synthetic construct
  misc_feature 1..681: note = Description of sequence: end mIgG1
  misc_feature 1..681: note = Description of sequence: mIL220 AA linker-mIgG1-mTL1A sequence component
  source 1..681: mol_type = other DNA
gtgcccagggattctggttctaagccttccatatctacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacaaaaccccgggaggagcagttcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaa

SEQ ID NO: 8 - skipped

SEQ ID NO: 9 - DNA, 567 bp, synthetic construct
  misc_feature 1..567: note = Description of sequence: end TL1A + stop
  misc_feature 1..567: note = Description of sequence: mIL220 AA linker-mIgG1-mTL1A sequence component
  source 1..567: mol_type = other DNA
cgggtccccggaaaagactgtatgcttcgggccataacagaagagagatctgagccttcaccacagcaagtttactcacctcccagaggcaagccgagagcacacctgacaattaagaaacaaaccccagcaccacatctgaaaaatcagctctctgctctacactgggaacatgacctagggatggccttcaccaagaacgggatgaagtacatcaacaaatccctggtgatcccagagtcaggagactatttcatctactcccagatcacattccgagggaccacatctgtgtgtggtgacatcagtcgggggagacgaccaaacaagccagactccatcaccatggttatcaccaaggtagcagacagctaccctgagcctgcccgcctactaacagggtccaagtctgtgtgtgaaataagcaacaactggttccagtccctctaccttggggccacgttctccttggaagaaggagacagactaatggtaaacgtcagtgacatctccttggtggattacacaaaagaagataaaactttctttggagctttcttgctataa

SEQ ID NO: 10 - skipped

SEQ ID NO: 11 - DNA, 1836 bp, synthetic construct
  misc_feature 1..1836: note = Description of sequence:
  misc_feature 1..1836: note = Description of sequence: mIL220 AA linker-mIgG1-mTL1A sequence
  source 1..1836: mol_type = other DNA
ctcgaggccaccatggacagcatgcagctcgcatcctgtgtcacattgacacttgtgctccttgtcaacagcgcacccacttcaagctctacttcaagctctacagcggaagcacagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaggaggcgggggttccggaggcgggggttcaggaggcgggggttccggaggcgggggttcactcgaggtgcccagggattctggttctaagccttccatatctacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacaaaaccccgggaggagcagttcaacagcactttccgttcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaagaattccgggtccccggaaaagactgtatgcttcgggccataacagaagagagatctgagccttcaccacagcaagtttactcacctcccagaggcaagccgagagcacacctgacaattaagaaacaaaccccagcaccacatctgaaaaatcagctctctgctctacactgggaacatgacctagggatggccttcaccaagaacgggatgaagtacatcaacaaatccctggtgatcccagagtcaggagactatttcatctactcccagatcacattccgagggaccacatctgtgtgtggtgacatcagtcgggggagacgaccaaacaagccagactccatcaccatggttatcaccaaggtagcagacagctaccctgagcctgcccgcctactaacagggtccaagtctgtgtgtgaaataagcaacaactggttccagtccctctaccttggggccacgttctccttggaagaaggagacagactaatggtaaacgtcagtgacatctccttggtggattacacaaaagaagataaaactttctttggagctttcttgctataa

SEQ ID NO: 12 - protein, 611 aa, synthetic construct
  REGION 1..611: note = Description of sequence:
  REGION 1..611: note = Description of sequence: mIL220 AA linker-mIgG1-mTL1A sequence
LEATMDSMQLASCVTLTLVLLVNSAPTSSSTSSSTAEAQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQGGGGSGGGGSGGGGSGGGGSLEVPRDSGSKPSISTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKEFRVPGKDCMLRAITEERSEPSPQQVYSPPRGKPRAHLTIKKQTPAPHLKNQLSALHWEHDLGMAFTKNGMKYINKSLVIPESGDYFIYSQITFRGTTSVCGDISRGRRPNKPDSITMVITKVADSYPEPARLLTGSKSVCEISNNWFQSLYLGATFSLEEGDRLMVNVSDISLVDYTKEDKTFFGAFLL

SEQ ID NO: 13 - protein, 170 aa, Mus musculus
  REGION 1..170: note = Description of Sequence: mIL-2
ATMDSMQLASCVTLTLVLLVNSAPTSSSTSSSTAEAQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 14 - protein, 227 aa, Mus musculus
  REGION 1..227: note = Description of Sequence: mIgG1
VPRDSGSKPSISTVPEVSSVFIFPPKPKDVLTITLTPKVTCVVVDISKDDPEVQFSWFVDDVEVHTAQTKPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - protein, 188 aa, Mus musculus
  REGION 1..188: note = Description of Sequence: mTL1A
RVPGKDCMLRAITEERSEPSPQQVYSPPRGKPRAHLTIKKQTPAPHLKNQLSALHWEHDLGMAFTKNGMKYINKSLVIPESGDYFIYSQITFRGTTSVCGDISRGRRPNKPDSITMVITKVADSYPEPARLLTGSKSVCEISNNWFQSLYLGATFSLEEGDRLMVNVSDISLVDYTKEDKTFFGAFLL

SEQ ID NO: 16 - protein, 227 aa, Homo sapiens
  REGION 1..227: note = Description of sequence: Human IgG1 (hinge+C2+C3; 229 aa)
CDKTHTCPPCPAPELLGGPSVFLFPPKPKDTLISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 194 aa, Homo sapiens
  REGION 1..194: note = Description of sequence: Human TL1A (full extracell. Domain; 191 aa)
EFRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 18 - protein, 153 aa, Homo sapiens
  REGION 1..153: note = Description of sequence: NM_000586.3 Homo sapiens interleukin 2 (IL2)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT